(19) United States
(12) Patent Application Publication
(10) Pub. No.: US 2025/0017559 A1
(43) Pub. Date: Jan. 16, 2025

(54) System and Method for Suggesting Catheter Parameters

(71) Applicant: Bard Access Systems, Inc., Salt Lake City, UT (US)

(72) Inventors: Gerald Blalock Denny, Salt Lake City, UT (US); Shayne Messerly, Kaysville, UT (US)

(21) Appl. No.: 18/221,318

(22) Filed: Jul. 12, 2023

(51) Int. Cl. A61B 8/08; A61B 8/00; A61B 8/12; G16H 10/60

(52) U.S. Cl. CPC A61B 8/0891; A61B 8/12; A61B 8/4483; A61B 8/461; A61B 8/5261; G16H 10/60

(57) ABSTRACT

A vasculature assessment device includes an imaging probe configured to acquire raw image data of a blood vessel of a patient and a device module having a console coupled with the imaging probe. Logic stored in memory device module determines blood vessel data from the raw image data and applies a trained machine learning model to the blood vessel data to determine suggested catheter parameters for catheter to be inserted within the blood vessel. A vasculature assessment system includes a plurality of the vasculature assessment devices and a computing system coupled with the vasculature assessment devices. Machine learning logic of the computing system performs a machine learning algorithm on historical catheter placement data sets to define the trained machine learning model.

### BACKGROUND

The placement of a catheter within a blood vessel often includes determining, prior to the catheter placement event, a catheter size and length that best aligns with blood vessel parameters of the patient. Typically the blood vessel parameters are ascertained using a blood vessel imaging device that is used to assist in the placement of the catheter. The complexity of such blood vessel imaging devices requires significant training on the part of the clinician to operate the blood vessel imaging device when placing the catheter to minimize risk to the patient. Due to the complex nature of the blood vessel imaging device, a similar significant level of training may be required to ascertain the blood vessel parameters even though the risk to the patient while ascertaining the blood vessel parameters pales in comparison to the risk associated with placement of the catheter. As such, there is a need for a system configured to ascertain the blood vessel parameters that can be operated by a clinician having less training than is required for a clinician to place the catheter.

Disclosed herein are devises, systems, and methods that address the forgoing.

### SUMMARY

Disclosed herein is a vasculature assessment device that, according to some embodiments, includes an imaging probe configured to acquire raw image data of a blood vessel of a patient and a device module having a console coupled with the imaging probe. The console includes a processor and a memory having logic stored thereon that, when executed by the processor performs operations that include (i) receiving the raw image data from the imaging probe; (ii) determining blood vessel data from the raw image data; (iii) applying a trained machine learning (ML) model to the blood vessel data to determine suggested catheter parameters pertaining to insertion of a catheter within the blood vessel; and (iv) depicting the suggested catheter parameters on a display of the device module.

In some embodiments, the blood vessel data include a diameter of the blood vessel and a depth of the blood vessel with respect to a skin surface, and in some embodiments, the suggested catheter parameters include a catheter size and a catheter length.

In some embodiments, the imaging probe is an ultrasound probe. In some embodiments, a head of the ultrasound probe includes a plurality of transducers, where all of the transducers are arranged in a single linear array.

In some embodiments, the imaging probe is configured to obtain the raw image data via A-mode ultrasound imaging technology in combination with near infrared (NIR) imaging technology.

In some embodiments, the operations further include determining an image of the blood vessel from the raw image data and depicting the image on the display.

In some embodiments, the suggested catheter parameters include a suggested catheter model, and in such embodiments, the operations include choosing the suggested catheter model from a list of catheter models stored in the memory.

In some embodiments, the operations further include communicating the suggested catheter parameters to an electronic medical record (EMR) system for inclusion in an EMR of the patient.

In some embodiments, the operations further include receiving the trained ML model from an external computing system and storing the trained ML model in the memory.

In some embodiments, the imaging probe, the device module, and the display are combined into a single unit.

Also disclosed herein is a vasculature assessment system that, according to some embodiments, includes a plurality of the vasculature assessment devices according to any of the vasculature assessment devices described above and a computing system coupled with the vasculature assessment devices. The computing system includes memory having ML logic stored thereon that, when executed by processors of the computing system, performs ML operations that include performing a ML algorithm on historical catheter placement data sets to define the trained ML model. The historical catheter placement data sets include blood vessel data sets received from the vasculature assessment devices and actual catheter parameter data sets that individually correspond with the blood vessel data sets.

In some embodiments, the computing system is communicatively coupled with an EMR system, and the ML operations include receiving the actual catheter parameter data sets from the EMR system.

In some embodiments, each blood vessel data set includes a diameter of a blood vessel of a respective patient and a depth of the blood vessel with respect to a skin surface of the respective patient, and in some embodiments, each actual catheter parameter data set includes a catheter size and a catheter length of a catheter inserted into the blood vessel of the respective patient. In some embodiments, each actual catheter parameter data set includes a catheter model.

Also disclosed herein is a system method of determining suggested catheter parameters that, according to some embodiments, includes (i) receiving a plurality of blood vessel data sets from a plurality of vasculature assessment devices; (ii) receiving a plurality of actual catheter parameter data sets from an electronic medical record (EMR) system, where each catheter parameter data set corresponds to a blood vessel data set; (iii) performing a machine learning (ML) algorithm on the blood vessel data sets and the actual catheter parameter data sets to train an ML model that relates the blood vessel data sets to the actual catheter parameter data sets; (iv) acquiring raw image data of a blood vessel from an instant patient utilizing one of the vasculature assessment devices; (v) determining instant blood vessel data from the raw image data; (vi) applying the (ML) model to the instant blood vessel data to determine suggested catheter parameters for a catheter to be inserted into the blood vessel; and (vii) depicting the suggested catheter parameters on a display of the one of the vasculature assessment devices.

In some embodiments of the system method, the blood vessel data include a diameter of the blood vessel and a depth of the blood vessel with respect to a skin surface, and the suggested catheter parameters include a catheter size and a catheter length.

In some embodiments of the system method, each vasculature assessment device include an ultrasound probe configured to acquire the raw image data, and in some embodiments, the ultrasound probe is configured to utilize A-mode ultrasound imaging to acquire the raw image data.

These and other features of the concepts provided herein will become more apparent to those of skill in the art in view of the accompanying drawings and following description, which describe particular embodiments of such concepts in greater detail.

### DRAWINGS

FIG. 1A illustrates a vasculature assessment device configured to determine suggested catheter parameters for a catheter to be inserted into the vasculature, in accordance with some embodiments.

FIG. 1B is a bottom view of an imaging module of the device of FIG. 1A, in accordance with some embodiments.

FIG. 2 illustrates a block diagram of a console of the device of FIG. 1A, in accordance with some embodiments.

FIG. 3 illustrates a cross-sectional side view of a blood vessel of a patient having a catheter inserted therein, in accordance with some embodiments.

FIG. 4 illustrates a vasculature assessment system configured to define a trained machine learning model of the device of FIG. 1A, in accordance with some embodiments.

FIG. 5 illustrates a block diagram of a system method for determining suggested catheter parameters for a catheter to be inserted into the vasculature of a patient, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. For example, the “systemic response” a used herein may include a systemic response of one type or a combination of systemic responses of multiple types.

The phrases “connected to,” “coupled with,” and “in communication with” refer to any form of interaction between two or more entities, including but not limited to physical, mechanical, electrical, magnetic, electromagnetic, fluid, wireless, and thermal interaction. Two components may be coupled with each other even though they are not in direct contact or communication with each other. For example, two components may be coupled with each other through an intermediate component.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

Unless defined otherwise, all technical and scientific terms used herein have the same meaning as commonly understood by those of ordinary skill in the art.

The terms “proximal” and “distal” refer to opposite ends of a medical device, including the devices disclosed herein. As used herein, the proximal portion of a medical device is the portion nearest a practitioner during use, while the distal portion is the portion at the opposite end. For example, the proximal end of a catheter is defined as the end closest to the practitioner during utilization of the catheter. The distal end is the end opposite the proximal end, along the longitudinal direction of the catheter.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

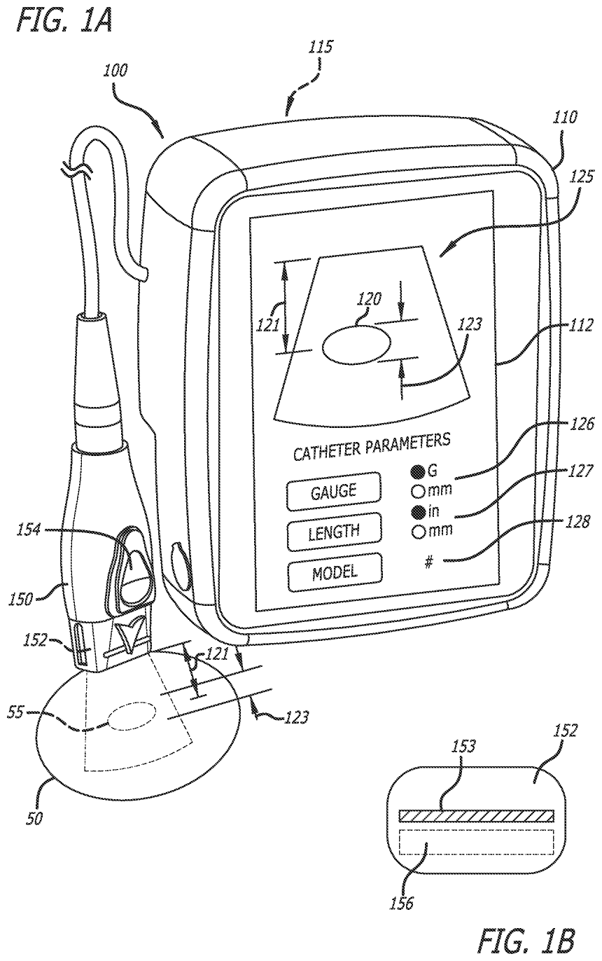

FIG. 1A illustrates a vasculature assessment device (device) 100 configured to determine suggested parameters of a vascular access catheter to be inserted into a blood vessel of patient, according to some embodiments. The device 100 is configured to assist a clinician in identifying and or preparing a catheter to be used to access the blood vessel without requiring significant training in the use of imaging systems. In some instances, a first minimally trained clinician may utilize the device 100 to determine catheter parameters, e.g., size, length and or model, for a catheter to be inserted into the blood vessel and second clinician may utilize a second imaging system (that requires the more significant training) to provide guiding and/or placing assistance during insertion of the catheter.

The device 100 generally includes an imaging probe 150 operatively coupled (e.g., via a wired or wireless connection) with a device module 110. The imaging probe 150 is configured to obtain/acquire raw image data of a blood vessel 55 of a patient 50. Logic of a console 115 or the device 100 determines blood vessel data from the raw image data, e.g., depth 121 of the blood vessel with respect to a skin surface of the patient and a diameter 123 of the blood vessel 55. The logic further determines from the blood vessel data suggested catheter parameters, e.g., a catheter size or gauge 126, a length 127 and/or catheter model 128 as described in more detail below. The device 100 notifies the clinician of the catheter parameters so that the clinician may identify, obtain, and/or prepare the catheter for insertion. In some instances, preparing the catheter includes trimming the catheter to a desired length.

The imaging probe 150 includes an imaging module 152 configured to obtain the raw image data. The imaging module 152 may include any suitable imaging technology capable of obtaining the image 125. In the illustrated embodiment, the imaging module 152 includes ultrasound technology. In other embodiments, the imaging module 152 may include imagining technology other than ultrasound. The imaging probe 150 may include control buttons 154 that enable the clinician operate the device 100 from the imaging probe 150. As also shown, the logic may determine from the raw image data an image 125 of the target area of the patient including a blood vessel image 120 of the blood vessel 55 and cause the image 125 to be depicted on the display 112.

FIG. 1B is a bottom end view of one embodiment of the imaging module 152 that utilizes ultrasound technology to obtain the image 125. In the illustrated embodiment, the imaging module 152 includes a plurality of ultrasound transducers 153 arranged in a liner array consistent with A-mode ultrasound imaging. The A-mode (i.e. one-dimensional) ultrasound technology provides a cost advantage over other two-dimensional ultrasound technologies. In some embodiments, the imaging module 152 may include a near infrared (NIR) imaging module 156 so that the imaging probe 150 may utilize A-mode ultrasound imaging in combination with NIR imaging to obtain the raw image data.

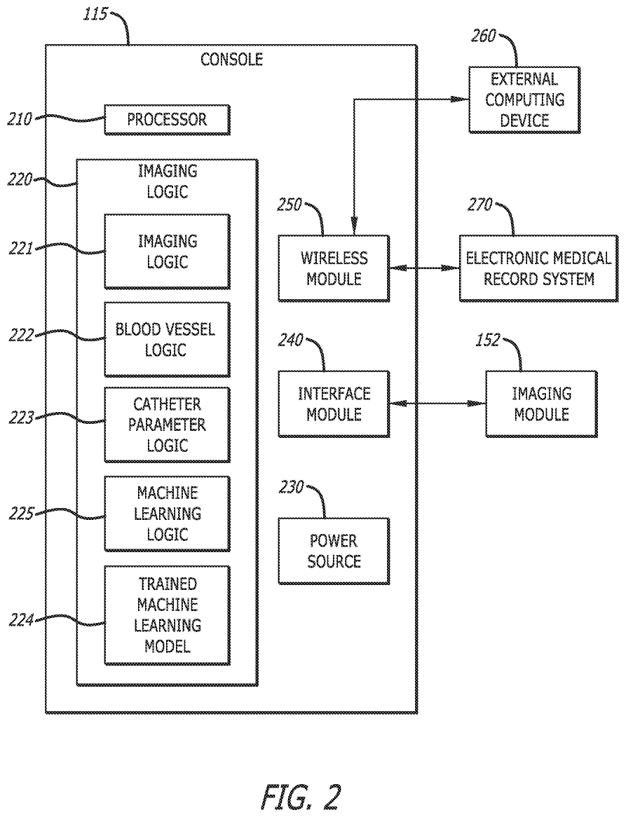

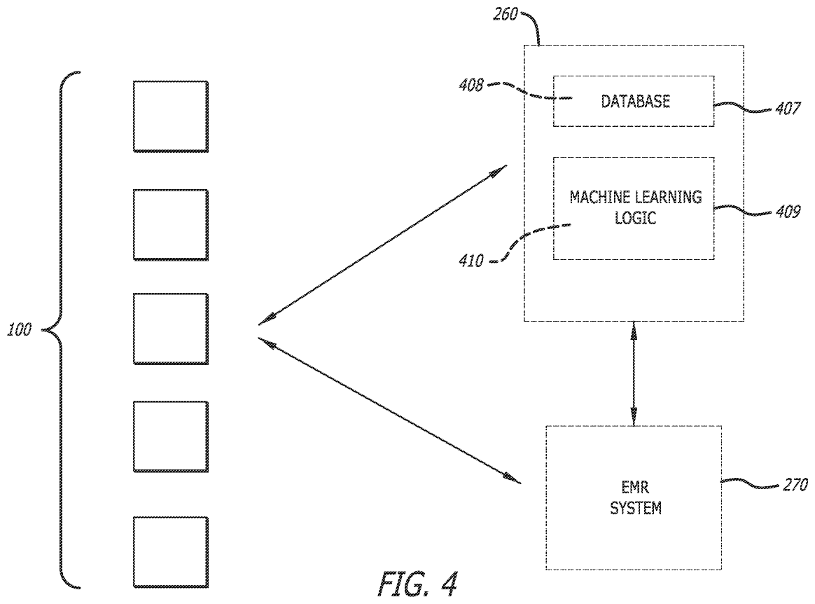

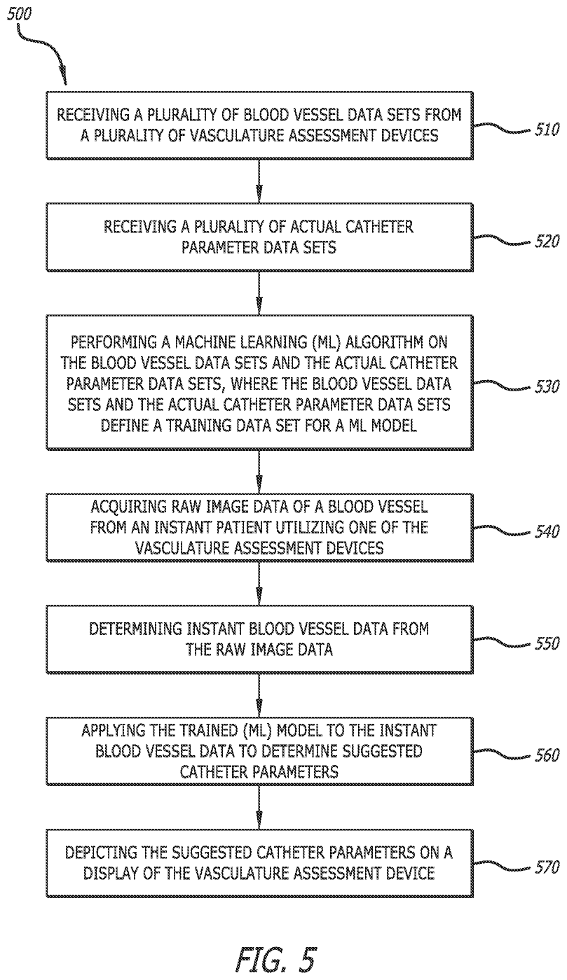

FIG. 2 illustrates a block diagram of the console 115, according to some embodiments. The console 115 is generally configured to govern the operation of the device 100. The console 115 includes a processor 210 and memory 220 (e.g., a non-transitory computer-readable medium) having logic modules stored thereon, such as imaging logic 221, a blood vessel logic 222, and catheter parameter logic 223, for example. Machine learning logic 225 and one or more trained machine learning models 224 may also be stored in the memory 220. Execution of and processing by the machine learning logic 225 and the one or more trained machine learning models 224 is discussed below with respect to at least FIGS. 4-5. The console 115 may include a wireless module 250 to facilitate wireless communication with an external computing device (or system) 260, and/or an electronic medical record (EMR) system 270 as further described below. The console 115 may be incorporated entirely into either the imaging probe 150 or the device module 110. Similarly, the console 115 may be incorporated partially into both the imaging probe 150 and the device module 110. In some embodiments, although not shown, the imaging probe 150 and the device module 110 may be combined into a single handheld unit. For example, the console 115 and the display 112 may integrated in the imaging probe 150.

The console 115 may include an interface module 240 configured to define operative coupling with the imaging module 152, such as converting analog signals to digital data, for example. The console 115 is powered via a power source 230. In some embodiments, the power source 230 may include an internal battery (e.g., a rechargeable battery) capable of providing sufficient power to operate the device 100 over a defined operational duration. Other power sources are also contemplated and therefore, included herein, such as an external facility power source, for example.

The imaging logic 221 is configured to acquire the raw image data (e.g., govern the operation of the imaging module 152). The imaging logic 221 may also determine the image 125 from the raw image data. The blood vessel logic 222 is configured to determine blood vessel data for the blood vessel 55 from the raw image data. The blood vessel data may include any parameters associated with the blood vessel 55 that may be helpful in identifying a catheter or catheter parameters suitable for insertion into the blood vessel 55. The blood vessel data may include a position and/or a shape of the blood vessel 55. Regarding position, the blood vessel data may include a depth 212 of the blood vessel 55 with respect to the skin surface. In some instances, the depth 212 may be related to the length of the catheter. Regarding shape of the blood vessel 55, the blood vessel data may include a diameter 123 of the blood vessel 55. In some instances, the blood vessel 55 may not be round (i.e., may include an oval or flattened shape). As such, the diameter 123 may be a minimum diameter of the blood vessel 55. The diameter 123 may be related to size/gauge (i.e., diameter) of the catheter to be inserted into the blood vessel 55.

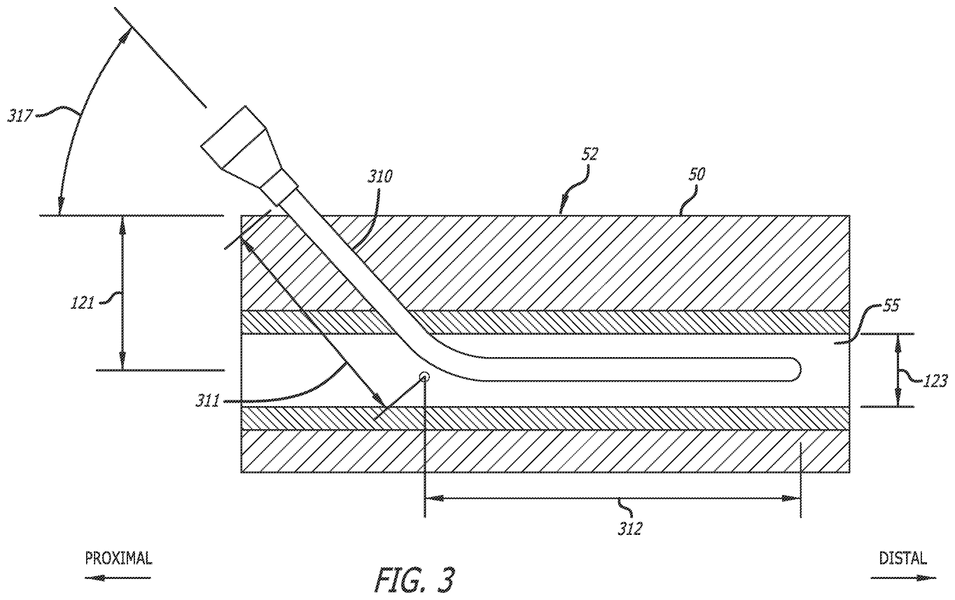

FIG. 3 illustrates a side cross-sectional view of a catheter 310 inserted into the blood vessel 55. Shown is the blood vessel 55 having the diameter 123 disposed at the depth 121 from the skin surface 52. The catheter 310 is inserted through tissue of the patient into the blood vessel 55. A proximal portion of the catheter 310 having a length 311 extends between the skin surface 52 and the blood vessel 55 at an insertion angle 317. A distal portion of the catheter 310 having a length 312 extends along the blood vessel 55. Accordingly a length of the catheter 310 may include a sum of the lengths 311, 312.

The catheter parameter logic 223 is configured to determine suggested catheter parameters based on the blood vessel data. In the illustrated embodiment, the catheter parameter logic 223 utilizes the trained machine learning (ML) model 224 to determine the suggested catheter parameters. More specifically, the catheter parameter logic 223 may apply the trained ML model 224 to the blood vessel data to determine the suggested catheter parameters. In accordance with the illustrated embodiment, the catheter parameter logic 223 may apply the trained ML model 224 to the blood vessel data to determine one or more of the catheter size (gauge), the catheter length or a catheter model from at least the depth of the blood vessel 121 and the diameter 123 of the blood vessel.

The catheter parameter logic 223 may cause the catheter size (gauge) 126, the catheter length 127 and/or the catheter model 128 to be depicted on the display 112. The catheter parameter logic 223 may also transmit the catheter size (gauge) 126, the catheter length 127 and/or the catheter model 128 to the EMR system 270 for inclusion in the EMR for the patient 50.

FIG. 4 illustrates a vasculature assessment system (system) 400, in accordance with some embodiments. The system 400 is generally configured to define the trained ML model 224, i.e., train the ML model 224. The system 400 generally includes a plurality of devices 100 coupled with a computing device 260. According to one embodiment, the computing device 260 may be an external computing device such as a network server for example. In some embodiments, the computing device 260 may be incorporated into the EMR system 270. In other embodiments, the computing device 260 may be incorporated into one or more of the devices 100.

The computing device 260 includes a database 407 and machine learning (ML) logic 409 stored on a non-transitory computer-readable medium. The ML logic 409 is configured to acquire historical catheter placement data sets to form a training data set 408 stored in the data base 407. The ML logic 409 is further configured to apply an ML algorithm 410 to the training data set 408 to define the trained ML model 224, where the ML logic 409 may be comprised of or configured to execute a plurality of ML algorithms 410 (e.g., predictive algorithms such as linear regression, logistic regression, classification and regression trees, Naïve Bayes, K-Nearest neighbors, etc.). The historical catheter placement data sets include blood vessel data sets and actual catheter parameter data sets that correspond individually (i.e., according to one to one relationship) with the blood vessel data sets. More specifically each blood vessel data set corresponds with an actual catheter parameter data set for a single catheter placement event.

The blood vessel data set for the catheter placement event includes the depth of the blood vessel and the diameter of the blood vessel. The actual catheter parameter data set includes the parameters of the catheter (e.g., size, length and/or model) that was actually placed within the patient as part of the catheter placement event. In some instances, the parameters of the catheter may be recorded in the EMR for the patient.

The computing device 260 may be coupled with the EMR system 270, and the ML logic 409 may acquire the actual catheter parameter data sets from the EMR system 270. The trained ML module 224 may be stored in the memory of the computing device 260. The ML logic 409 may transmit or communicate the trained ML model 224 to the devices 100 for storage in the memory 220.

FIG. 5 is a block diagram of a system method 500 of determining suggested catheter parameters that, according to some embodiments, includes all or any subset of the following actions, steps, or processes. Each block illustrated in FIG. 5 represents an operation of the method 500 performed by a vasculature assessment system and/or a vascular assessment device disclosed herein, and typically as a result of execution of one or more logic modules disclosed herein as well as deployment of specific machines or devices, such as the vasculature assessment device 100 and the vasculature assessment system 400. The method 500 may include receiving a plurality of blood vessel data sets from a plurality of vasculature assessment devices (block 510). The blood vessel data sets may include a diameter of the blood vessel and a depth of the blood vessel with respect to a skin surface as determined from raw image data acquired from a patient via one of the vasculature assessment devices. The vasculature assessment device may include an ultrasound probe configured to acquire the raw image data, and in some embodiments, the ultrasound probe is configured to utilize A-mode ultrasound imaging acquire the raw image data.

The method 500 method may further include receiving a plurality of actual catheter parameter data sets (block 520), which may be acquired from an electronic medical record (EMR) system, where each catheter parameter data set corresponds to a blood vessel data set. The method 500 method may further include performing a machine learning (ML) algorithm on the blood vessel data sets and the actual catheter parameter data sets, where the blood vessel data sets and the actual catheter parameter data sets define a training data set for a ML model (block 530), and where a result of performing the machine learning (ML) algorithm is a trained ML model that relates the blood vessel data sets to the actual catheter parameter data sets.

The method 500 method may further include acquiring raw image data of a blood vessel from an instant patient utilizing one of the vasculature assessment devices (block 540). The method 500 method may further include determining instant blood vessel data from the raw image data (block 550). The method 500 method may further include applying the trained (ML) model to the instant blood vessel data to determine suggested catheter parameters (block 560), where the suggested catheter parameters pertain to a catheter to be inserted within the blood vessel. The suggested catheter parameters may include a catheter size and a catheter length. The method 500 method may further include depicting the suggested catheter parameters on a display of the one of the vasculature assessment devices (block 570).

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A vasculature assessment device, comprising:
an imaging probe configured to acquire raw image data of a blood vessel of a patient; and
a device module having a console coupled with the imaging probe, the console including a processor and a memory having logic stored thereon that, when executed by the processor, performs operations that include:
receiving the raw image data from the imaging probe;
determining blood vessel data from the raw image data;
applying a trained machine learning (ML) model to the blood vessel data resulting in suggested catheter parameters pertaining to insertion of a catheter within the blood vessel; and
depicting the suggested catheter parameters on a display of the device module.

2. The device according to claim 1, wherein the blood vessel data include a diameter of the blood vessel and a depth of the blood vessel with respect to a skin surface.

3. The device according to claim 1, wherein the suggested catheter parameters include a catheter size and a catheter length.

4. The device according to claim 1, wherein the imaging probe is an ultrasound probe.

5. The device according to claim 1, wherein:
a head of the ultrasound probe includes a plurality of transducers, and
all of the transducers are arranged in a single linear array.

6. The device according to claim 1, wherein the imaging probe is configured to obtain the raw image data via A-mode ultrasound imaging technology in combination with near infrared (NIR) imaging technology.

7. The device according to claim 1, wherein the operations further include:
determining an image of the blood vessel from the raw image data; and
depicting the image on the display.

8. The device according to claim 1, wherein the suggested catheter parameters include a catheter model, the operations including choosing the catheter model from a list of catheter models stored in the memory.

9. The device according to claim 1, wherein the operations further include communicating the suggested catheter parameters to an electronic medical record (EMR) system for inclusion in an EMR for the patient.

10. The device according to claim 1, wherein the operations further include receiving the trained ML model from an external computing system and storing the trained ML model in the memory.

11. The device according to claim 1, wherein the imaging probe, the device module, and the display are combined into a single unit.

12. A vasculature assessment system, comprising:
a plurality of the vasculature assessment devices, each vasculature assessment device comprising:
an imaging probe configured to acquire raw image data of a blood vessel of a patient; and
a device module having a console coupled with the imaging probe, the console including a processor and a memory having logic stored thereon that, when executed by the processor performs operations that include:
receiving the raw image data from the imaging probe;
determining blood vessel data from the raw image data;
applying a trained machine learning (ML) model to the blood vessel data to determine suggested catheter parameters pertaining to catheter to be inserted within the blood vessel; and
depicting the suggested catheter parameters on a display of the device module; and
a computing system coupled with the vasculature assessment devices, the computing system including memory having ML logic stored thereon that, when executed by processors, performs ML operations that include performing a ML algorithm on historical catheter placement data sets to define the trained ML model, wherein the historical catheter placement data sets include:
blood vessel data sets received from the vasculature assessment devices; and
actual catheter parameter data sets,
wherein each actual catheter parameter data set corresponds to a blood vessel data set in a one to one relationship.

13. The system according to claim 12, wherein the computing system is communicatively coupled with an EMR system, the ML operations including receiving the actual catheter parameter data sets from the EMR system.

14. The system according to claim 12, wherein each blood vessel data set includes a diameter of a blood vessel of a respective patient and a depth of the blood vessel with respect to a skin surface of the respective patient.

15. The system according to claim 14, wherein each actual catheter parameter data set includes a catheter size and a catheter length of a catheter inserted into the blood vessel of the respective patient.

16. The system according to claim 15, wherein each actual catheter parameter data set further includes a catheter model.

17. A system method of determining suggested catheter parameters, comprising:
receiving a plurality of blood vessel data sets from a plurality of vasculature assessment devices;
receiving a plurality of actual catheter parameter data sets from an electronic medical record (EMR) system, each catheter parameter data set corresponding to a blood vessel data set;
performing a machine learning (ML) algorithm on the blood vessel data sets and the actual catheter parameter data sets to train a ML model that relates the blood vessel data sets to the actual catheter parameter data sets;
acquiring raw image data of a blood vessel from an instant patient utilizing one of the vasculature assessment devices;
determining instant blood vessel data from the raw image data;
applying the (ML) model to the instant blood vessel data to determine suggested catheter parameters for a catheter to be inserted within the blood vessel; and
depicting the suggested catheter parameters on a display of the one of the vasculature assessment devices.

18. The system method according to claim 17, wherein:
the blood vessel data include a diameter of the blood vessel and a depth of the blood vessel with respect to a skin surface, and
the suggested catheter parameters include a catheter size and a catheter length.

19. The system method according to claim 17, wherein each vasculature assessment device includes an ultrasound probe configured to acquire the raw image data.

20. The system method according to claim 17, wherein the ultrasound probe is configured to utilize A-mode ultrasound imaging to acquire the raw image data.
